(19) United States
(12) Patent Application Publication
(10) Pub. No.: US 2010/0136694 A1
(43) Pub. Date: Jun. 3, 2010

(54) Cone-Shaped Adapter for a Gene Gun

(75) Inventors: Jay D. Potts, Columbia, SC (US); Conrad Michael Gore, West Columbia, SC (US)

Correspondence Address: DORITY & MANNING, P.A., POST OFFICE BOX 1449, GREENVILLE, SC, 29602-1449, US

(73) Assignee: University of South Carolina, Columbia, SC (US)

(21) Appl. No.: 12/327,079

(22) Filed: Dec. 3, 2008

(51) Int. Cl. C12N 15/87; C12M 1/00

(52) U.S. Cl. 435/455; 435/285.3

(57) ABSTRACT

A cone-shaped adapter for attachment to a muzzle of a gene gun is generally provided. The cone-shaped adapter comprises sidewalls that taper from a base plane at one end and to an apex aperture at an opposite end and a fitting. The sidewalls form a base angle of from about 85° to about 45° with the base plane. The fitting is attached to the sidewalls at the base plane and is configured to connect the cone-shaped adapter to the muzzle of the gene gun. Methods of using the cone-shaped adapter with a gene gun are also provided.

### BACKGROUND OF THE INVENTION

High-density micron-size particles accelerated to high speeds can penetrate deep inside live tissues have been used to effectively deliver genetic material. This method of ballistic delivery is often called “biolistics” and the device for shooting particles is referred to as a “gene gun.” Biolistics is a direct physical method of introducing nucleic acids into cells. For example, nucleic acids or other biological molecules are coated onto high-density gold or tungsten microparticles and then are accelerated to high velocity by a helium pulse and driven through cell walls and membranes into the target cell. This technology is especially useful for cells and tissues that are refractory to other transformation methods, such as certain plants.

A hand-held commercially available version of the gene gun, Helios® by BioRad of Hercules, Calif. uses about 1 μm in diameter particles made of gold or tungsten, which are accelerated by a short pulse of a high-speed flow of helium. Although the carrier particle size has been selected to minimize the cell injury, the high-speed jet of He emerging from the gun muzzle can produce severe damage to soft tissue. In order to slow the flow of He, the nozzle (or barrel) expands toward the end, the target is usually shot from a significant distance, and sometimes a mesh filter is placed between the muzzle and the target. Although there is little data on the actual speeds of the particles launched from the Helios® gene gun, all of those measures result in deceleration of the particles and reduction of their penetration depth. However, the expanded end of the gene gun and relatively large distance used between the end of the gun and the target creates a significant margin of error for the entry point of the particles on the target. As such, the particles are typically shot at the target in a “shotgun blast” like manner without precise control over the targeting of the particles. The loss of precise control over the entry point of the particles can impede the ability to deliver genetic material to a specific cell or specific areas of the cell (e.g., the cell nucleus), which can define extremely small targets.

As such, a need currently exists for modifications to existing gene gun technologies that enable precise control of the bio-particles shot from the gene gun to enable the particles to be targeted precisely for delivery to the desired area of the cell.

### SUMMARY OF THE INVENTION

Objects and advantages of the invention will be set forth in part in the following description, or may be obvious from the description, or may be learned through practice of the invention.

In general, the present disclosure is directed toward a cone-shaped adapter for attachment to a muzzle of a gene gun. The cone-shaped adapter comprises sidewalls that taper from a base plane at one end and to an apex aperture at an opposite end and a fitting. The sidewalls form a base angle of from about 85° to about 45° with the base plane. The fitting is attached to the

sidewalls at the base plane and is configured to connect the cone-shaped adapter to the muzzle of the gene gun.

The present disclosure is also directed to a gene gun including the cone-shaped adapter attached to its muzzle.

The present disclosure is also directed to methods of using the cone-shaped adapter with a gene gun.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures, in which:

FIG. 1 shows a side view of an exemplary cone-shaped adapter of the present invention;

FIG. 2 shows a perspective view of the exemplary cone-shaped adapter of FIG. 1; and

FIG. 3 shows the exemplary cone-shaped adapter of FIG. 1 attached to the end of a muzzle of a gene gun.

Repeat use of reference characters in the present specification and drawings is intended to represent the same or analogous features or elements of the present invention.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

In general, the present disclosure is directed to an adapter configured to be attached to the end of a gene gun to focus the particles exiting the end of the gene gun. The focused particles shot from the gene gun, through the presently disclosed adapter, can enable the delivery of nucleic acids or other biological material to specifically targeted areas of a cell. Thus, the “shotgun blast” of particles that would normally exit the end of the gene gun (that is not equipped with the presently disclosed adapter) can be avoided, and the delivery of particles can be precisely delivered to the target cell or area of the cell. The particles can therefore be delivered to the target cell, instead of multiple cells (when desired).

The adapters can be configured to attach to any gene guns, particularly commercially available gene guns. Particularly suitable commercially available gene guns are sold under the name Helios® Gene Gun (Bio-Rad Laboratories, Inc., Hercules, Calif.). These gene guns shoot about 1 μm in diameter particles made of gold or tungsten, which are accelerated by a short pulse of a high-speed flow of helium. In order to slow the flow of He, the muzzle expands toward the end, the target is usually shot from a significant distance, and sometimes a mesh filter is placed between the muzzle and the target. The adapter of the present disclosure is configured to attach to the expanded end of the muzzle of the gene gun.

The adapter is generally cone-shaped and tapers away from the muzzle of the gene gun. The cone-shape of the adapter focuses the particles exiting the muzzle of the gene gun to increase the accuracy of the particles shot from the gun. The apex of the cone defines an aperture such that the cone-shaped adapter essentially defines a hollow truncated cone. Thus, the particles exiting the gene gun are focused into a narrow, targeted beam by the cone and allowed to exit the adapter through the aperture at the apex of the cone.

The cone shape defined by the adapter is generally a right circular cone, where right means that the axis passes through the center of the base at right angles to its plane and circular means that the base is a circle. However, configurations other than a right, circular cone can be utilized to focus the particles exiting the muzzle of the gene gun within the scope of the present application.

Only the sidewalls of the cone-shaped adapter are physically defined by the adapter (i.e., the base of the cone and the apex of the cone are defined by apertures) allowing the particles to flow freely through the inside of the adapter. Specifically, the particles can enter the inside of the cone-shaped adapter through the base plane aperture, be focused by the inner surfaces of the cone sidewalls, and exit the apex aperture toward the target.

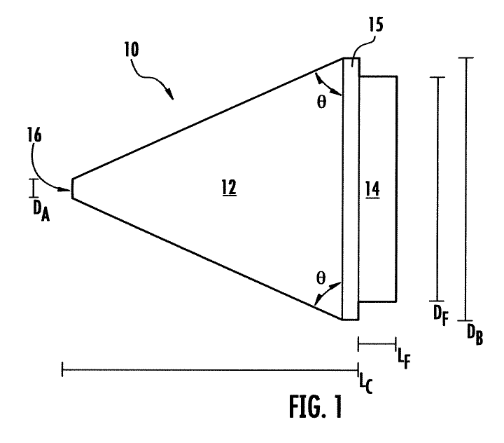

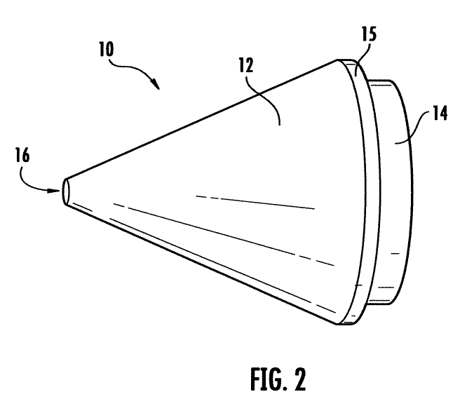

FIGS. 1 and 2 show an exemplary cone-shaped adapter 10 having sidewalls 12. The cone-shaped adapter 10 also defines a fitting 14 for attachment to the muzzle of the gene gun. The sidewalls 12 of the cone-shaped adapter 10 taper away from the fitting 14 until the sidewalls 12 are truncated to form an apex aperture 16. The base plane also defines an aperture that is at least the size of the muzzle of the gene gun to allow the particles exiting the muzzle of the gene gun to flow into the inside space defined by the cone-shaped adapter 10. Thus, the particles exiting the muzzle of the gene gun can be focused by the inner surface of the sidewalls 12 such that the particles exit the apex aperture 16 as a more focused beam toward the target.

The base plane of the cone-shaped adapter 10 is generally defined by the end of the sidewalls 12 opposite the apex aperture 16, which form a circle having a base diameter D_{B}. The base diameter D_{B} is generally sized to match the particular gene gun to which the cone-shaped adapter 10 will be attached. Specifically, the diameter of the muzzle of the gene gun and the base diameter D_{B} defined by end of the sidewalls 12 can be nearly identical (i.e., within about 5% of the diameter of the muzzle of the gene gun). For example, when using a Helios® gene gun that has a muzzle diameter of one inch, the base diameter D_{B} is also one inch (or between about 95% and about 105% of one inch).

The sidewalls 12 form a base angle θ with the base plane. The base angle θ of the sidewalls 12 to the base plane can vary as desired, as long as the sidewalls 12 taper towards each other (i.e., the base angle θ on either side is less than 90°). In most embodiments, however, the base angle θ of the sidewalls 12 is between about 85° and about 45°, such as between about 75° and about 55°. If the angle is too sharp (e.g., less than 45°), then the particles exiting the muzzle of the gene gun may ricochet off the inner surfaces of the sidewalls 12 and return towards the muzzle of the gene gun.

Generally, the base angle θ is constant around the base in order to form a right circular cone. The base angle θ may vary slightly however as long as the effectiveness of the cone-shaped adapter 10 is not severely hindered.

The shape of the apex diameter D_{A} is generally a circle defined by the end of the sidewalls 12 of the cone-shaped adapter 10. The apex aperture 16 can have any apex diameter D_{A} that is smaller than the muzzle diameter of the gene gun but larger than the particle size in order to focus the particles exiting the gene gun in a more targeted manner. In most embodiments, the apex diameter D_{A} is significantly smaller than the base diameter D_{B} (which generally corresponds to the muzzle diameter of the gene gun), such as less than about 10% of the base diameter D_{B}. The apex diameter D_{A} can be, for example, from about 5% to about 0.1% of the base diameter D_{B}, such as from about 3.5% to about 1% of the base diameter D_{B}. For instance, referring to the embodiment having a base diameter D_{B} of one inch, the apex diameter D_{A} can be less than about 0.1 inch, such as from about 0.05 to about 0.001 inch and from about 0.035 inch to about 0.01 inch.

The cone length L_{C} will generally vary according to the base angle θ, the base diameter D_{B}, and the aperture diameter D_{A}. In most embodiments, the cone length L_{C} can be measured as a function of the base diameter D_{B}, and can be from about 50% to about 500% of the base diameter D_{B}, such as from about 75% to about 200% of the base diameter D_{B}. Thus, the cone length L_{C} can typically vary from about 0.5 inch to about 5 inches, such as from about 0.75 to about 2 inches, when attached to a muzzle having a diameter of one inch. For instance, the cone length L_{C} can be about one inch, when the other dimensions are as follows: a base diameter D_{B} of one inch, a base angle θ of about 64°, and an apex diameter D_{A} of about 0.025 inch.

The cone-shaped adapter 10 may be constructed from any material that is durable enough to withstand the particles being shot into the inner surface of the sidewalls 12. For example, the cone-shaped adapter 10 can be constructed of a metal (e.g., steel, stainless steel, titanium, aluminum, etc., and alloys thereof), ceramic material, plastics (e.g., thermoplastic or thermoset plastics), etc. The material selected should avoid scratching or etching after repeated use (especially on the inner surface of the sidewalls 12), and should be able to focus the particles into the targeted area through the apex aperture 16.

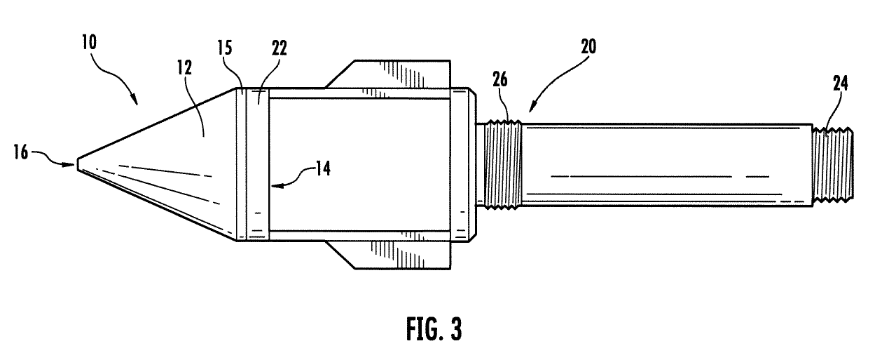

The cone-shaped adapter 10 also defines a fitting 14 configured to attach the cone-shaped adapter 10 to the muzzle of the gene gun. The fitting 14 can essentially be shaped as the tubular walls of a hollow cylinder. In one embodiment, the outer surface of the fitting 14 is configured for attachment to the inner surface of the muzzle of the gene gun (either through a snug fit or a threaded fit). Referring to FIG. 3, the cone-shaped adapter 10 is attached to the muzzle 20 by inserting the fitting 14 of the cone-shaped adapter 10 into the nozzle end 22 of the muzzle 20. The muzzle 20 also defines securing threading 24, 26 for attachment to the gene gun. FIG. 3 represents a configuration suitable for use with the Helios® gene gun; however, it should be noted that one of ordinary skill in the art would be able to vary the attachment of the cone-shaped adapter 10 to the muzzle of various gene guns without undue experimentation.

When configured for insertion within the muzzle 20, the fitting 14 can generally define a fitting diameter D_{F}, defined by the outer surface of the fitting 14, that corresponds to the inner diameter of the muzzle of the gene gun. The fitting length L_{F} can be sufficient to steady the fitting 14 within the muzzle of the gene gun during use.

The fitting 14 can extend through the base plane of the cone-shaped adapter 10 to be attached (e.g., welded) to the inner surface of the sidewalls 12. Alternatively, the portion of the base plane between the base end of the sidewalls 12 and the outer surface of the fitting 14 can be an extension of the sidewalls 12.

Other fitting configurations can be used, such as an outer fitting configured to attached to the outer surface of the muzzle of the gene gun. The present disclosure is also directed to a method of focusing particles shot from a muzzle of a gene gun into a cell. According to the method, the cone-shaped adapter is attached to the muzzle of the gene gun. The particles are shot out of the muzzle of the gene gun through the muzzle of the gene gun, into the cone-shaped adapter (focusing the particles to the target), out of the apex aperture, and into the cell (or cells).

The cone-shaped adapter 10 may optionally include a base tube section 15 positioned between the base end of the sidewalls 12 and the fitting 14. The base tube section 15, when present, effectively extends the length of the cone-shaped adapter 10 without changing the direction of the particles (until the particles enter the cone-shaped section). The base tube section 15 may also facilitate the production of the cone-shaped adapter 10 (e.g., facilitate milling, etc.).

As discussed above, the gene gun can be any system that accelerates particles to be shot into a cell via a gas. In general, a “gene gun” is a device that delivers DNA to cells by microprojectile bombardment with extremely high speed delivery (sometime referred to as a “Biolistic Particle Delivery System”). The Helios® Gene Gun uses Biolistic® particle bombardment where DNA- or RNA-coated gold particles are loaded into the gun and one pulls the trigger for delivery. A high pressure helium pulse delivers the coated gold particles into virtually any target cell or tissue. The particles carry the DNA so that one does not have to remove cells from tissue in order to transform the cells. One model of the Helios gene gun system, 220-240 V, is used for biolistic particle delivery of biomaterials into cells. This handheld device employs an adjustable helium pulse to sweep DNA-, RNA-, and other biomaterial-coated gold microcarriers from the inner wall of a small plastic cartridge directly into target cells. This system has a 2 square-centimeter target area, and uses a pressure range of 100-600 psi. The system includes the Helios gene gun, helium hose assembly, helium regulator, tubing prep station, syringe kit, tubing cutter, and Helios gene gun optimization kit.

The payload is an elemental particle of a heavy metal coated with plasmid DNA. Any particle typically shot out of the muzzle of the gene gun can be used in accordance with the cone-shaped adapter described herein. These particles are typically metal particles (e.g., gold, tungsten, etc.) coated with the desired bio-material (e.g., DNA, proteins, viruses, and the like). The size of the particles are typically on the micrometer or nanometer scale (e.g., from about 10 nm to about 10 micrometers in diameter).

### EXAMPLE

A cone-shaped adapter was formed, such as shown in FIGS. 1 and 2, and attached to the end nozzle of the muzzle of a Helios® gene gun as shown in FIG. 3. The nozzle end 22 of the muzzle 20 had an outer diameter of 1 inch and an inner diameter of 0.885 inch.

The cone-shaped adapter was formed from stainless steel and had a fitting diameter D_{F} of 0.884 inch, a fitting length L_{F} of 0.2 inch, a cone length L_{C} of one inch, a base diameter D_{B} of one inch, a base angle θ of about 64° and an apex diameter D_{A} of 0.025 inch.

When inserted into the end of the muzzle of the gene gun, the adapter effectively focused the particles exiting the gene gun to precisely target the particles to the desired cell.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. A cone-shaped adapter for attachment to a muzzle of a gene gun, the cone-shaped adapter comprising:
sidewalls that taper from a base plane at one end and to an apex aperture at an opposite end, wherein the sidewalls form a base angle of from about 85° to about 45° with the base plane; and
a fitting attached to the sidewalls at the base plane, wherein the fitting is configured to connect the cone-shaped adapter to the muzzle of the gene gun.

2. The cone-shaped adapter of claim 1, wherein the base angle is from about 75° to about 55°.

3. The cone-shaped adapter of claim 1, wherein the base plane defines a circle having a base diameter, and wherein the apex aperture has a diameter of less than about 10% of the base diameter.

4. The cone-shaped adapter of claim 3, wherein the apex aperture has a diameter of from about 5% to about 0.1% of the base diameter.

5. The cone-shaped adapter of claim 3, wherein the apex aperture has a diameter of from about 3.5% to about 1% of the base diameter.

6. The cone-shaped adapter of claim 1, wherein the cone-shaped adapter defines a cone length from the base plane to the aperture apex, wherein the cone length is from about 0.5 inch to about 5 inches.

7. The cone-shaped adapter of claim 6, wherein the cone length is from about 0.75 to about 2 inches.

8. The cone-shaped adapter of claim 1, wherein the cone shaped adapter is constructed from a metal.

9. The cone-shaped adapter of claim 8, wherein the metal comprises stainless steel.

10. The cone-shaped adapter of claim 1, wherein the cone-shaped adapter is constructed from a ceramic material.

11. The cone-shaped adapter of claim 1, wherein the cone-shaped adapter is constructed from a plastic material.

12. The cone-shaped adapter of claim 1, wherein the apex aperture has a diameter of less than about 0.1 inch.

13. The cone-shaped adapter of claim 1, wherein the apex aperture has a diameter of from about 0.05 inch to about 0.001 inch.

14. The cone-shaped adapter of claim 1, wherein the apex aperture has a diameter of from about 0.035 inch to about 0.01 inch.

15. The cone-shaped adapter of claim 1, wherein the sidewalls define a right circular cone.

16. The cone-shaped adapter of claim 1, wherein fitting is configured for insertion within the muzzle of the gene gun.

17. The cone-shaped adapter of claim 16, wherein the fitting is configured to fit snugly within an inner surface of the muzzle of the gene gun.

18. The cone-shaped adapter of claim 16, wherein the fitting is threaded to screw into the muzzle of the gene gun.

19. A gene-gun comprising a muzzle allowing particles to be shot from the gene gun out of the muzzle propelled via a gas; and
a cone-shaped adapter attached to the muzzle of the gene gun, wherein the cone-shaped adapter comprises sidewalls that taper from a base plane at one end and to an apex aperture at an opposite end and a fitting attached to the sidewalls and configured for attachment to the muzzle, wherein the sidewalls form an base angle of from about 85° to about 45° with the base plane

20. A method of focusing particles shot from a muzzle of a gene gun into a cell, the method comprising
attaching a cone-shaped adapter to the muzzle of the gene gun, wherein the cone-shaped adapter comprises sidewalls that taper from a base plane at one end and to an apex aperture at an opposite end and a fitting attached to the sidewalls and configured for attachment to the muzzle, wherein the sidewalls form an base angle of from about 85° to about 45° with the base plane; and
shooting particles out of the muzzle of the gene gun through the muzzle of the gene gun, into the cone-shaped adapter, out of the apex aperture, and into the cell.
